(19) United States
(12) Patent Application Publication
(10) Pub. No.: US 2010/0330596 A1
(43) Pub. Date: Dec. 30, 2010

(54) LIPOPROTEIN SURFACTANT

(76) Inventor: Lindy Jane Murphy, Surbiton (GB)

Correspondence Address: ROCHE DIAGNOSTICS OPERATIONS INC., 9115 Hague Road, Indianapolis, IN, 46250-0457, US

(21) Appl. No.: 12/636,320

(22) Filed: Dec. 11, 2009

Related U.S. Application Data
(63) Continuation of application No. PCT/GB08/01996, filed on Jun. 11, 2008.

(30) Foreign Application Priority Data
Jun. 11, 2007 (GB) 0711236.0

(51) Int. Cl. C12Q 1/61; C12M 1/40; C12Q 1/60

(52) U.S. Cl. 435/11; 435/287.1; 435/4

(57) ABSTRACT

Surfactants are provided which are particularly useful for carrying out cholesterol and triglyceride tests. These surfactants have particularly fast kinetics of response to cholesterol, cholesterol ester and triglyceride in all lipoprotein particles. Cholesterol or triglyceride sensors incorporating these surfactants therefore provide reliable measurements of the total cholesterol or triglyceride content of a sample in a short period of time. Also provided are a sensor comprising the subject surfactants for determining the amount of triglyceride and/or cholesterol in a sample, as well as methods for determining the amount of cholesterol and/or triglyceride in a sample, the method comprising: contacting the sample with a surfactant as defined above; and determining the amount of cholesterol and/or triglyceride present.

### CLAIM OF PRIORITY

The present application is a continuation application based on and claiming priority to PCT Application No. PCT/GB08/001,996, filed Jun. 11, 2008, which claims the priority benefit of British to Application No. GB 0711236.0, filed Jun. 11, 2007, each of which are hereby incorporated by reference in their entireties.

### TECHNICAL FIELD OF THE INVENTION

The present invention relates to a sensor for determining the amount of triglyceride or cholesterol in a sample and to a method for carrying out such a determination.

### BACKGROUND

Triglycerides and cholesterols are major components of lipoproteins found in the blood. Triglycerides are found largely in the very low density lipoproteins (VLDL) and chylomicrons (CM), whilst cholesterol is found largely in the high density lipoproteins (HDL) and low density lipoproteins (LDL). Cholesterol and triglycerides occur to some degree in all of the lipoprotein fractions in the blood.

To carry out an effective test on a blood or plasma sample, therefore, the lipoproteins must first be broken down to liberate the triglycerides and cholesterol (including free cholesterol and cholesterol esters). This is generally achieved using a surfactant. However, many surfactants act at different rates with the different lipoprotein fractions. To ensure that the triglyceride or cholesterol test detects all of the analyte present in the sample, it is therefore necessary to allow the surfactant to react with the sample for a sufficient period of time to enable all of the available triglyceride or cholesterol to be liberated.

As test kits become available which enable cholesterol and triglyceride testing to be carried out in the home or by medical practitioners in a clinic, the demand for rapid results in these tests is significantly increased. Ideally, a test kit would provide an accurate result in a matter of seconds, or a few minutes. To achieve this aim, there is a need for a fast acting surfactant which is able to rapidly break down all lipoprotein fractions in the blood.

### SUMMARY

This object and others that will be appreciated by a person of ordinary skill in the art have been achieved according to the embodiments of the present invention disclosed herein. In one embodiment, the present invention comprises a group of surfactants which are particularly useful for carrying out cholesterol and triglyceride tests. These surfactants have particularly fast kinetics of response to cholesterol, cholesterol ester and triglyceride in all lipoprotein particles. Cholesterol or triglyceride sensors incorporating these surfactants therefore provide reliable measurements of the total cholesterol or triglyceride content of a sample in a short period of time.

The present invention accordingly provides a sensor for determining the amount of triglyceride and/or cholesterol in a sample, the sensor comprising:

(a) a surfactant of formula (1)

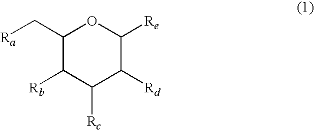

wherein
each of Rₐ, R_{b}, R_{c}, R_{d} and Rₑ is independently —OH, C₁-C₄ alkoxy or a group of formula —OCONH(CH₂)_{m′}—CH₃, —OCO(CH₂)_{m′}—CH₃, O(CH₂)ₘ—CH₃, —S(CH₂)_{m″}—CH₃, —O(CH₂)ₙ-A, —S(CH₂)ₙ-A, —OCO(CH₂)ₘ—CH₃ or —NHCO(CH₂)ₘ—CH₃, wherein m is from 4 to 20, m′ is from 4 to 20, m″ is from 4 to 6 or from 8 to 20, n is from 0 to 10 and A is a C₃-C₈ cycloalkyl group or a phenyl group,
with the proviso that at least one of the groups Rₐ, R_{b}, R_{c}, R_{d} and Rₑ is not —OH or C₁-C₄ alkoxy; and

(b) an enzyme reagent for measuring triglycerides and/or an enzyme reagent for measuring cholesterol.

Further embodiments of the present invention relate to a method for determining the amount of cholesterol and/or triglyceride in a sample, the method comprising: contacting the sample with a surfactant as defined above; and determining the amount of cholesterol and/or triglyceride present.

The sensor of the present invention is for determining the amount of triglyceride and/or cholesterol in a sample. For the avoidance of doubt, this means that the sensor is for determining the total amount of triglyceride and/or the total amount of cholesterol in a sample. Total cholesterol will include the cholesterol present in both HDL and LDL fractions in a sample (as well as, of course, free cholesterol and cholesterol present in any other form in the sample, for example as cholesterol esters).

The invention is to be explained in more detail by the following figures and examples.

### BRIEF DESCRIPTION OF THE DRAWINGS

The following detailed description of the embodiments of the present invention can be best understood when read in conjunction with the following drawings, where like structure is indicated with like reference numerals and in, which:

FIGS. 1-6 are graphs plotting the sensor output (measured current, Iox (nA)) vs total cholesterol concentration ([TC] (mM)) for a number of plasma samples using various sensors of the invention.

In order that the present invention may be more readily understood, reference is made to the following detailed descriptions and examples, which are intended to illustrate the present invention, but not limit the scope thereof.

### DETAILED DESCRIPTION OF EMBODIMENTS OF THE PRESENT INVENTION

The following descriptions of the embodiments are merely exemplary in nature and are in no way intended to limit the present invention or its application or uses.

The sensor of the invention can be used to measure the cholesterol or triglyceride levels of any sample containing lipoproteins. Typically, an analysis can be carried out on any body fluids, human or animal, typically for example, on whole blood, serum or plasma samples. Preferred samples for use in the invention are serum and plasma. Where measurements are to be carried out on whole blood, the method may include the additional step of filtering the blood to remove red blood cells.

The surfactant is a saccharide which may be a D-saccharide or L-saccharide, with D-saccharides being preferred. Both the α and β isomers can be used. In one embodiment, β isomers are preferred.

The surfactant has the general formula (1):

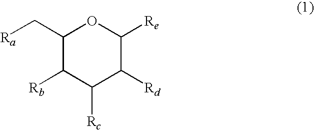

In the formula (1), each of Rₐ, R_{b}, R_{c}, R_{d} and Rₑ is independently —OH, C₁-C₄ alkoxy or a group of formula —OCONH(CH₂)_{m′}—CH₃, —OCO(CH₂)_{m′}—CH₃, O(CH₂)ₘ—CH₃,

—S(CH₂)_{m″}—CH₃, —O(CH₂)ₙ-A, —S(CH₂)ₙ-A, —OCO(CH₂)ₘ—CH₃ or —NHCO(CH₂)ₘ—CH₃, wherein m is from 4 to 20, m′ is from 4 to 20, m″ is from 4 to 6 or from 8 to 20, n is from 0 to 10 and A is a C₃-C₈ cycloalkyl group or a phenyl group. At least one of the groups Rₐ, R_{b}, R_{c}, R_{d} and Rₑ is not —OH or C₁-C₄ alkoxy.

Preferably, one or two, most preferably one, of the groups Rₐ, R_{b}, R_{c}, R_{d} and Rₑ is a group of formula —OCONH(CH₂)_{m′}—CH₃, —OCO(CH₂)_{m′}—CH₃, —SCONH(CH₂)_{m′}—CH₃,

—SCO(CH₂)_{m′}—CH₃, O(CH₂)ₘ—CH₃, —S(CH₂)_{m″}—CH₃, —O(CH₂)ₙ-A, —S(CH₂)ₙ-A,

—OCO(CH₂)ₘ—CH₃ or —NHCO(CH₂)ₘ—CH₃, the remaining groups being —OH or C₁-C₄ alkoxy, preferably —OH.

It is preferred that the surfactant of general formula (1) has the general formula (I):

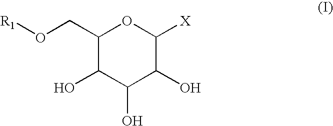

wherein:

i) R₁ is a group of formula —CONH(CH₂)_{m′}—CH₃ or —CO(CH₂)_{m′}—CH₃ wherein m′ is from 4 to 20; and X is —OH or C₁-C₄ alkoxy; or

ii) R₁ is hydrogen or C₁-C₄ alkyl; and X is a group of formula

—O(CH₂)ₘ—CH₃, —S(CH₂)_{m″}—CH₃, —O(CH₂)ₙ-A, —S(CH₂)ₙ-A,

—OCO(CH₂)ₘ—CH₃ or —NHCO(CH₂)ₘ—CH₃ wherein m is from 4 to 20, m″ is from 4 to 6 or from 8 to 20, n is from 0 to 10 and A is a C₃-C₈ cycloalkyl group or a phenyl group.

In a first embodiment of the present invention, the surfactant has the general formula (I) and R₁ is a group of formula —CONH(CH₂)_{m′}—CH₃ or —CO(CH₂)_{m′}—CH₃, wherein m′ is from 4 to 20, X is —OH or C₁-C₄ alkoxy. In this embodiment, m′ is preferably from 3 to 10, for example from 4 to 9. Particularly preferred is m′ being 6 or 7, most preferably 6. Furthermore, in this embodiment X is preferably —OH or methoxy, most preferably methoxy. Within this embodiment, it is preferred that R₁ has the formula —CONH(CH₂)_{m′}—CH₃. Specific preferred surfactants in this embodiment are methyl-6-O—(N-alkylcarbamoyl)-α-D-glucopyranosides, where the alkyl group contains from 5 to 10 carbon atoms. These surfactants are available as Anameg-5, Anameg-6, Anameg-7, Anameg-8, Anameg-9 and Anameg-10 from Anatrace, where the index refers to the total length of the alkyl chain (i.e., the group (CH₂)_{m′}—CH₃). A particularly preferred surfactant is O—(N-heptylcarbamoyl)-α-D-glucopyranoside, which is Anameg-7.

In a second embodiment of the invention, the surfactant has the general formula (I) and R₁ is hydrogen or C₁-C₄ alkyl, X is a group of formula —O(CH₂)ₘ—CH₃,

—S(CH₂)_{m″}—CH₃, —O(CH₂)ₙ-A, —S(CH₂)ₙ-A, —OCO(CH₂)ₘ—CH₃ or —NHCO(CH₂)ₘ—CH₃, wherein m is from 4 to 20, m″ is from 4 to 6 or from 8 to 20, n is from 0 to 10 and A is a C₃-C₈ cycloalkyl group or a phenyl group. In this second embodiment, R₁ is preferably hydrogen or methyl, most preferably hydrogen. m is preferably from 5 to 9, for example from 6 to 8. Furthermore, m″ is preferably from 4 to 6. Still further, n is preferably from 0 to 5, for example from 0 to 3. The C₃-C₈ cylcoalkyl group may be a cyclopropyl, cyclobutyl, cyclopentyl, cyclohexyl, cycloheptyl or cyclooctyl group, with cyclopentyl and cyclohexyl, in particular cyclohexyl, being preferred. The group A is preferably C₃-C₈ cylcoalkyl group, most preferably cyclohexyl. When A is a cycloalkyl group, n is preferably at, least 1, for example from 1 to 5. When A is a phenyl group, n is preferably from 0 to 3.

In the second embodiment of the invention, one preferred group of surfactants is where X is a group of formula —O(CH₂)ₘ—CH₃, —S(CH₂)_{m″}—CH₃, —OCO(CH₂)ₘ—CH₃ or —NHCO(CH₂)ₘ—CH₃. Specific preferred surfactants are n-alkyl-β-D-glucopyranosides, where the alkyl group contains from 6 to 10 carbon atoms, such as n-octyl-β-D-glucopyranoside (known as OGP, available from Anatrace) and n-nonyl-β-D-glucopyranoside (known as NGP, also available from Anatrace). Other specific preferred surfactants are n-alkyl-α-D-glucopyranosides, n-alkyl-β-D-thioglucopyranosides, n-alkyl-β-D-galactopyranosides and n-alkyl-β-D-mannopyranosides, where the alkyl group contains from 6 to 10 carbon atoms: for example, n-octyl-α-D-glucopyranoside, n-heptyl-β-D-thioglucopyranoside, n-octyl-β-D-galactopyranoside and n-octyl-β-D-mannopyranoside.

An alternative preferred group of surfactants in the second embodiment of the invention is where X is a group of formula —O(CH₂)ₙ-A or —S(CH₂)ₙ-A. Specific preferred surfactants are 3-cyclohexyl-1-methyl-β-D-glucoside (known as Cyglu-1), 3-cyclohexyl-1-ethyl-β-D-glucoside (known as Cyglu-2) and particularly 3-cyclohexyl-1-propyl-β-D-glucoside (known as Cyglu-3), all available from Anatrace. When A is a phenyl group n is preferably from 0 to 8, for example from 0 to 4. Further specific preferred surfactants are phenyl β-D-glucopyranoside, phenyl β-D-galactopyranoside and phenylethyl β-D-galactopyranoside.

Particularly preferred surfactants in the present invention are methyl-6-O—(N-heptylcarbamoyl)-D-glucopyranoside and 3-cyclohexyl-1-propyl-D-glucoside.

The surfactant is preferably of the formula (Ia), (Ib) or (Ic):

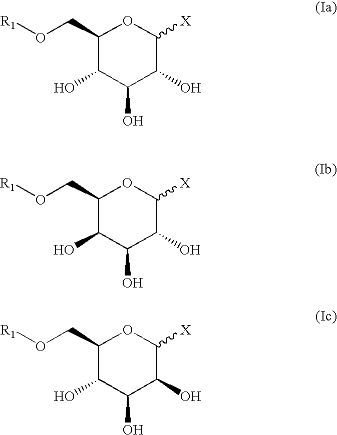

Thus, the surfactant of formula (Ia) may preferably be a glucopyranose (formula (Ia)), a galactopyranose (formula (Ib)) or a mannopyranose (formula (Ic)).

In a further embodiment of the invention, the surfactant is of formula (Ix) or (II)

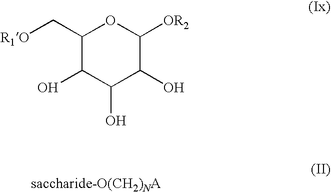

wherein R₁′ is a group of formula —CONH(CH₂)_{M}—CH₃ wherein M is from 6 to 20, R₂ is hydrogen or methyl, A is a C₃-C₈ cycloalkyl group and N is from 1 to 10.

In this further embodiment, the surfactant may be a 6-O-carbamoyl saccharide of formula (Ix). Preferably, the surfactant of formula (Ix) is a glucoside. M is typically from 6 to 10, for example 6 or 7. Preferred surfactants include methyl-6-O—(N-heptylcarbamoyl)-α-D-glucopyranoside (Anameg-7, available from Anatrace).

Also in this further embodiment, the surfactant may be of formula (II) and thus derived from reaction of a saccharide with an alcohol of formula A-(CH₂)_{N}—OH. Thus, the compound of formula (II) consists of a saccharide molecule wherein at least one, e.g. one, —OH group is replaced with a —O(CH₂)_{N}-A group. The saccharide may be a mono, di or trisaccharide, for example glucose, maltose, maltotriose or sucrose. Monosaccharides, in particular glucose, are preferred. The surfactant is preferably a 1-glucoside. The cycloalkyl group A may be a cyclopropyl, cyclobutyl, cyclopentyl, cyclohexyl, cycloheptyl or cyclooctyl group, with cyclopentyl and cyclohexyl, in particular cyclohexyl, being preferred. N is preferably from 1 to 8, typically from 2 to 6. Preferred surfactants include 3-cyclohexyl-1-propyl-β-D-glucoside (Cyglu-3, available from Anatrace).

Also in this further embodiment, the surfactant used in the invention may be a surfactant of formula (Ixx):

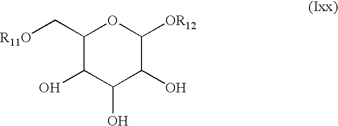

wherein either R₁₁ is a group of formula —CONH(CH₂)_{M}—CH₃ and R₁₂ is hydrogen or methyl; or R₁₁ is hydrogen and R₁₂ is —(CH₂)_{N}-A, wherein A, N and M are as defined above.

In the present invention, it is preferred that the surfactant possesses a particular HLB value. HLB value is a well-known parameter in the context of surfactants and describes the hydrophilicity of a surfactant. The HLB value of a particular surfactant can be obtained readily, using an NMR methodology as described in Raboron et al., International Journal of Pharmaceutics, Vol 99, 1993, p 23-36. This reference shows that HLB value can be calculated as:

$$HLB = \frac{60 H}{2 + H} , \\ wherein$$

$$H = \frac{{A}_{hydrophilic}}{{A}_{total}} ,$$

wherein A_{hydrophilic} is the NMR integral of the chemical shifts for hydrophilic groups and Aₜₒₜₐₗ is the NMR integral of all chemical shifts in the surfactant.

HLB values for certain specific surfactants of the invention obtained using the above-described method are as follows:

| Surfactant | HLB |
|---|---|
| Anameg-5 | 13.0 |
| Anameg-6 | 12.0 |
| Anameg-7 | 11.2 |
| Anameg-8 | 10.5 |
| Anameg-9 | 9.5 |
| Anameg-10 | 8.7 |
| β-Cyglu-1 | 11.0 |
| β-Cyglu-2 | 10.1 |
| β-Cyglu-3 | 9.5 |
| α-Cyglu-3 | 9.4 |
| n-octyl-β-D-glucopyranoside | 9.5 |
| n-octyl-β-D-mannopyranoside | 9.5 |
| n-octanoyl-β-D-glucopyranoside | 8.0 |

In the present invention, it is preferred that the surfactant has an HLB value of from 5 to 16, for example from 7 to 14.

The surfactants of the invention are commercially available products or can be produced by the skilled person using standard synthetic techniques. Further examples of surfactants which can be employed in the present invention, and details regarding the synthesis of these surfactants, can be found in U.S. Pat. No. 5,223,411 and U.S. Pat. No. 5,763,586.

The surfactant is typically provided in such an amount that when mixed with the sample to be tested the concentration of surfactant in the mixture of sample with the surfactant and any other reagents used is at least 10 mM, preferably at least 20 mM, for example at least 25 mM.

The surfactants described herein may be used alone or in combination. Thus, the sensor of the present invention may comprise one surfactant of formula (1), for example a surfactant of formula (I), as a sole surfactant, or a surfactant of formula (1) along with one or more further surfactants. The one or more further surfactants may also fall within the general formula (1). In one embodiment, the sensor comprises two or more, for example up to five, surfactants, which are different, but each have the general formula (1) and preferably the general formula (1). For example, the sensor may comprise two such surfactants. Preferred combinations are:
Anameg-7 and n-nonyl-β-D-glucopyranoside (NGP)
Cyglu-3 and n-nonyl-β-D-glucopyranoside (NGP)
Anameg-7 and Cyglu-3.

Also preferred are combinations comprising at least two of Anameg-7, Cyglu-3 and OGP.

The triglyceride or cholesterol that is liberated by the surfactant of the invention reacts with the enzyme reagent in order to provide a quantitative measure of the amount of triglyceride or cholesterol that is present in the sample. The enzyme reagent used is not particularly limited and any enzyme suitable for carrying out a triglyceride or cholesterol test may be used. For example, in the case of cholesterol, the enzyme reagent may comprise cholesterol dehydrogenase or cholesterol oxidase. In the triglyceride test, examples of enzyme reagents that can be used are glycerol dehydrogenase and glycerol kinase in combination with glycerol phosphate oxidase.

Any commercially available forms of cholesterol dehydrogenase or glycerol dehydrogenase may be employed. For instance, the cholesterol dehydrogenase is, for example, from the Nocardia species and the glycerol dehydrogenase is, for example, from the Cellulomonas species. The dehydrogenase may be used in an amount of from 0.1 to 100 mg per ml of sample, preferably from 0.5 to 45 mg per ml.

Reaction of lipoproteins with the surfactant will typically liberate cholesterol both in its free form and in the form of cholesterol esters. In the cholesterol test, therefore, the enzyme reagent will typically comprise a cholesterol ester hydrolysing reagent in order to break down the esters into free cholesterol. The cholesterol ester hydrolysing reagent may be any reagent capable of hydrolysing cholesterol esters to cholesterol. The reagent should be one which does not interfere with the reaction of cholesterol with cholesterol dehydrogenase and any subsequent steps in the assay. Preferred cholesterol ester hydrolysing reagents are enzymes, for example cholesterol esterase and lipases. A suitable lipase is, for example, a lipase from a pseudomonas or Chromobacterium viscosum species. The cholesterol ester hydrolysing, reagent may be used in an amount of from 0.1 to 25 mg per ml of sample, for example from 0.1 to 20 mg per ml of sample, preferably from 0.5 to 25 mg per ml, such as 0.5 to 15 mg per ml.

In the triglyceride test, a glycerol enzyme is typically used to determine the triglyceride content. The triglycerides which are liberated from the lipoproteins must therefore first be broken down to glycerol before reaction with the glycerol dehydrogenase. This is typically achieved by including in the enzyme reagent a triglyceride hydrolysing reagent. Any reagent which hydrolyses triglycerides to glycerol may be used as long as it does not interfere with the activity of the dehydrogenase enzyme. Lipases and esterases are suitable examples of triglyceride hydrolysing reagents. The lipases described above as the cholesterol ester hydrolysing reagent are also appropriate for use in hydrolysing triglycerides. The triglyceride hydrolysing reagent may be used in an amount of from 0.1 to 100 mg per ml of sample, for example from 0.1 to 70 mg per ml of sample, preferably from 0.5 to 25 mg per ml, such as 0.5 to 15 mg per ml. In one embodiment, the triglyceride hydrolysing reagent is used in an amount of from 0.1 to 25 mg per ml of sample, for example from 0.1 to 20 mg per ml of sample.

Each of the enzymes may contain additives such as stabilisers or preservatives. Further, each of the enzymes may be chemically modified.

Further reagents may be present in the sensor of the invention as required to carry out the determination of the amount of cholesterol or triglyceride which reacts with the enzyme reagent. Additives such as stabilizers, buffers and excipients may also be used. Reagents to activate the enzymes may also be added. For example ammonium chloride may be used to activate glycerol dehydrogenase.

The surfactant may be added to the sample prior to addition of the other reagents or simultaneously with the addition of the other reagents. In a preferred embodiment, the enzyme reagent and surfactant are present in a single reagent mixture which is combined with the sample in a single step. In a particularly preferred embodiment, the method involves a single step of contacting the sample with reagents, so that only a single reagent mixture need be provided.

The sensor of the invention may determine the cholesterol or triglyceride content of the sample by any appropriate technique. For example, peroxidase and a colour forming agent may be used to determine the hydrogen peroxide produced by reaction of an analyte with an oxidase enzyme. The skilled person in the art would be familiar with such methods. In a sensor for triglyceride detection making use of an oxidase system, the following reagents could, for example, be used: a lipase or cholesterol esterase, glyercol kinase, glycerol-3-phosphate oxidase, peroxidase and a mediator. In a sensor for cholesterol detection making use of an oxidase system, the following reagents could, for example, be used: a lipase or cholesterol esterase, cholesterol oxidase, peroxidase and a mediator. Particular examples of these reagents that would be suitable for use in such sensors are explained in more detail below.

In a preferred embodiment, an, electrochemical analysis is used and this embodiment will be described in further detail below. It is to be understood, however, that the present invention is not intended to be limited to electrochemical analyses.

In the electrochemical analysis, the amount of cholesterol or triglyceride which has reacted with the enzyme reagent is determined by measuring an electrochemical response occurring at an electrode. In this embodiment, the sample is typically reacted with the surfactant, the enzyme reagent, a coenzyme capable of interacting with the enzyme reagent, and a redox agent which is capable of being oxidised or reduced to form a product which can be electrochemically detected at an electrode. The mixture of sample and reagents is contacted with a working electrode of an electrochemical cell so that redox reactions occurring can be detected. A potential is applied across the cell and the resulting electrochemical response, typically the current, is measured.

In this preferred embodiment, the amount of cholesterol is measured in accordance with the following assay:

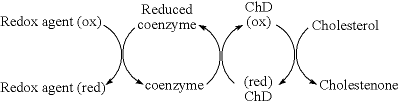

where ChD is cholesterol dehydrogenase.

Similarly, for a triglyceride sensor, the amount of triglyceride present may be determined in accordance with the following assay:

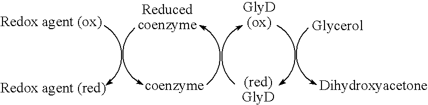

where GlyD is glycerol dehydrogenase. In either assay, the amount of reduced redox agent produced by the assay is detected electrochemically. Additional reagents may also be included in this assay if appropriate.

Typically, the sample contacts all of the reagents in a single step. Therefore, the sensor typically comprises a reagent mixture which contains all of the required reagents and which can easily be contacted with the sample in order to carry out the assay. The reagent mixture typically comprises the surfactant at a concentration of from 10 to 500 mM, preferably from 25 to 200 mM, in particular at least 50 mM or at least 75 mM. The hydrolysing reagent is typically present in an amount of from 0.1 to 25 mg, preferably from about 0.5 to 20 mg per ml of sample and the dehydrogenase in an amount of from 0.1 to 100 mg, preferably from 0.5 to 45 mg per ml of sample.

The amounts of each reagent are specified herein in terms of the concentration in the reagent mixture, or in terms of the mass per ml of reagent mixture. It is not, however, essential that the reagent mixture be provided in the form of a solution. It may alternatively be provided in dried form, for example it may be freeze dried. In these embodiments, the amounts of reagent described herein refer to the concentrations or mass in a solution or suspension of the reagent mixture prior to drying.

Typically the coenzyme is NAD⁺ or an analogue thereof. An analogue of NAD⁺ is a compound having structural characteristics in common with NAD⁺ and which also acts as a coenzyme for cholesterol dehydrogenase or glycerol dehydrogenase. Examples of NAD⁺ analogues include APAD (Acetyl pyridine adenine dinucleotide); TNAD (Thio-NAD); AHD (acetyl pyridine hypoxanthine dinucleotide); NaAD (nicotinic acid adenine dinucleotide); NHD (nicotinamide hypoxanthine dinucleotide); and NGD (nicotinamide guanine dinucleotide). The coenzyme is typically present in the reagent mixture in an amount of from 1 to 20 mM, for example from 3 to 15 mM, preferably from 5 to 10 mM.

Typically, the redox agent should be one which can be reduced in accordance with the assay shown above. In this case, the redox agent should be one which is capable of accepting electrons from a coenzyme (or from a reductase as described below) and transferring the electrons to an electrode. The redox agent may be a molecule or an ionic complex. It may be a naturally occurring electron acceptor such as a protein or may be a synthetic molecule. The redox agent will typically have at least two oxidation states.

Preferably, the redox agent is an inorganic complex. The agent may comprise a metallic ion and will preferably have at least two valencies. In particular, the agent may comprise a transition metal ion and preferred transition metal ions include cobalt, copper, iron, chromium, manganese, nickel, osmium or ruthenium. The redox agent may be charged, for example it may be cationic or alternatively anionic. An example of a suitable cationic agent is a ruthenium complex such as Ru(NH₃)₆³⁺. An example of a suitable anionic agent is a ferricyanide complex such as Fe(CN)₆³⁻.

Examples of complexes which may be used include Cu(EDTA)²⁻, Fe(CN)₆³⁻, Fe(CN)₅(O₂CR)³⁻, Fe(CN)₄(oxalate)³⁻, Ru(NH₃)₆³⁺, Ru(acac)₂(Py-3-CO₂H)(Py-3-CO₂) (herein after referred to as RuAcac) and chelating amine ligand derivatives thereof (such as to ethylenediamine), Ru(NH₃)₅(py)³⁺, ferrocenium and derivatives thereof with one or more of groups such as —NH₂, —NHR, —NHC(O)R, and —CO₂H substituted into one or both of the two cyclopentadienyl rings. Preferably the inorganic complex is Fe(CN)₆³⁻, Ru(NH₃)₆³⁺, Ru(acac)₂(Py-3-CO₂H)(Py-3-CO₂) or ferrocenium monocarboxylic acid (FMCA). Ru(NH₃)₆³⁺ and Ru(acac)₂(Py-3-CO₂H)(Py-3-CO₂) are preferred.

The redox agent is typically present in the reagent mixture in an amount of from 10 to 200 mM, for example from 20 to 150 mM, preferably from 30 to 100 mM, or up to 80 mM.

In a preferred embodiment, the reagent mixture used in the electrochemical assay additionally comprises a reductase. The reductase typically transfers two electrons from the reduced. NAD or analogue thereof and transfers two electrons to the redox agent. The use of a reductase therefore provides swift electron transfer.

Examples of reductases which can be used include diaphorase and cytochrome P450 reductases, in particular, the putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductases. Preferred reductases for use in the present invention include diaphorase and putidaredoxin reductases.

The reductase may be a recombinant protein or a naturally occurring protein which has been purified or isolated. The reductase may have been mutated to improve its performance such as to optimise the speed at which it carries out the electron transfer or its substrate specificity.

The reductase is typically present in the reagent mixture in, an amount of from 0.5 to 100 mg/ml, for example from 1 to 50 mg/ml, 1 to 30 mg/ml or from 2 to 20 mg/ml.

In a preferred embodiment of the invention, the general scheme of the cholesterol assay is as follows:

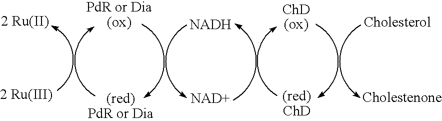

and the general scheme of the triglyceride assay is as follows:

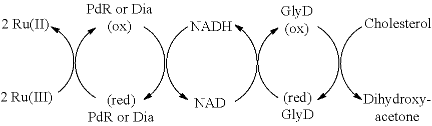

PdR—is putidaredoxin reductase

Dia—is diaphorase

ChD—is cholesterol dehydrogenase

GlyD—is glycerol dehydrogenase.

It will be well known to those persons skilled in the art that the dehydrogenase can be replaced by an oxidase with consequent changes to the cascade to enable measurement of peroxide or enzyme mediated electrochemistry.

The reagent mixture optionally contains one or more additional components, for example excipients and/or buffers and/or stabilisers. Excipients are preferably included in the reagent mixture, as well known in the art, in order to stabilize the mixture and optionally, where the reagent mixture is dried onto the device of the invention, to provide porosity in the dried mixture. Examples of suitable excipients include sugars such as mannitol, inositol and lactose, glycine and PEG. Buffers may also be included to provide the required pH for optimal enzyme activity. For example, a Tris buffer (pH9) may be used. Stabilisers may be added to enhance, for example, enzyme stability. Examples of suitable stabilisers are amino acids, e.g. glycine, and ectoine.

The sensor of the invention typically comprises a sensing device for measuring the amount of cholesterol or triglyceride which reacts with the oxidase or dehydrogenase. In a preferred embodiment, the sensor is for the electrochemical measurement of the cholesterol or triglyceride content. In this embodiment, the sensor includes an electrochemical cell having at least two electrodes. The cell may be a two electrode system having a working electrode and a counter electrode which also acts as a pseudo reference electrode. Alternatively, the cell may be a three electrode system having a working electrode, a reference electrode and a counter electrode. In a preferred embodiment the working electrode of the cell is a microelectrode, for example a microband electrode having a width of no more than 50 μm. Typically, the electrodes do not carry a coating layer comprising a water-soluble cellulose derivative (for example, carboxymethyl cellulose, ethylcellulose or hydroxypropyl cellulose) or anon-substituted water-soluble saccharide (for example, glucose, fructose, trehalose, sucrose, lactose or maltose).

The sensor typically also comprises a measuring unit, which includes a power supply for supplying a potential across the cell and a measuring instrument for measuring the resulting electrochemical response, typically the current across the cell.

Typically, the surfactant and enzyme reagent, and any further reagents required, are mixed together as a single reagent mixture which is suspended/dissolved in a suitable liquid (e.g. water or buffer) and provided to the sensor. The reagent mixture is then typically dried in position. This step of drying the material into/onto the sensor helps to keep the material in the desired position. Drying may be carried out, for example, by air-drying, vacuum drying, freeze drying or oven drying (heating), preferably by freeze drying. The reagent mixture is typically located in the vicinity of the electrodes, such that when the sample contacts the reagent mixture, contact with the electrodes also occurs.

The sensor may optionally comprise a membrane through which the sample to be tested passes prior to contact with the reagent mixture. The membrane may, for example, be used to filter out components such as red blood cells, erythrocytes and/or lymphocytes. Suitable filtration membranes, including blood filtration membranes, are known in, the art. Examples of blood filtration membranes are Presence 200 and PALL BTS SP300 of Pall filtration, Whatman VF2, Whatman Cyclopore, Spectral NX and Spectral X. Fibreglass filters, for example Whatman VF2, can separate plasma from whole blood and are suitable for use where a whole blood specimen is supplied to the device and the sample to be tested is plasma.

Alternative or additional membranes may also be used, including those which have undergone a hydrophilic or hydrophobic treatment prior to use. Other surface characteristics of the membrane may also be altered if desired. For example, treatments to modify the membrane's contact angle in water may be used in order to facilitate flow of the desired sample through the membrane. The membrane may comprise one, two or more layers of material, each of which may be the same or different. For example, conventional double layer membranes comprising two layers of different membrane materials may be used.

Appropriate devices for use in the present invention include those described in WO 2003/056319 and WO 2006/000828.

In the embodiments described above, the sensor of the invention is for the detection of triglyceride or cholesterol. However, the skilled person will appreciate that a single sensor could be used to provide a measurement of both the triglyceride and the cholesterol contents of a sample. In one embodiment, this is achieved by including two electrochemical cells in the sensor, one adapted for measurement of the cholesterol content of the sample, and one adapted for measurement of the triglyceride content of the sample. In the sensors described in WO 2003/056319 and WO 2006/000828, this can be achieved by providing the appropriate reagents for the cholesterol test to one electrochemical cell in the form of a receptacle, and providing the appropriate reagents for the triglyceride test to a second electrochemical cell in the form of a receptacle. The sensor can then be subjected to a freeze drying process so that the reagent mixtures are dried into position in their appropriate electrochemical cells. In this embodiment, the two reagent mixtures are fixed into localized positions so that the two assays can be carried out side by side without interfering with one another.

In the method of the present invention, a sample to be tested is contacted with the surfactant described herein and is further contacted with one or more other reagents to enable the cholesterol or triglyceride content to be measured. Typically, the sample is contacted with the reagent mixture described herein and the cholesterol or triglyceride content is measured electrochemically.

Sufficient time must be allowed for the reagent mixture to mix with the sample and for reaction to occur (a “wet-up” period) prior to taking the measurement. Where a plasma sample is used with sensors containing freeze dried reagents, a time of approximately 20 to 30 seconds elapses between contact of the sample with the surfactant and enzyme reagent and initiation of the test. This wet-up period may be as short as 20 seconds or even 15 seconds, but may be up to 45 seconds or 2 minutes. This short wet-up period is sufficient to allow the surfactant of the invention to break down all types of lipoprotein product present in the sample and to enable the liberated cholesterol or triglyceride to react with the enzyme reagent. Where whole blood is used, additional time may be required to allow for blood cell removal. For example, the whole blood sample may be provided to the sensor and a period of 4 or 5 minutes provided for blood cell removal and up-take/reaction with the reagent mixture.

In the electrochemical determination, the reagent mixture is typically present in the electrochemical cell prior to sample addition. Addition of the sample to the cell initiates the above wet-up period and application of a potential occurs after the wet-up period has elapsed. In an alternative embodiment, sample is mixed with the reagents off the electrode and added to the cell with immediate application of potential.

The electrochemical response is measured within a period of 10 seconds to 5 minutes after application of a sample. Typically, the electrochemical response is determined at least 0.5 minutes, for example at least 1 minute after application of a sample. In a preferred embodiment, the electrochemical response is determined at least 1.5 minutes, preferably at least 2 minutes after application of a sample.

Typically, where Ru(II) is the product to be detected at the working electrode, the potential applied to the cell is from 0.1V to 0.3V. Preferred applied potential is 0.15V. (All voltages mentioned herein are quoted against a Ag/AgCl reference electrode, with 0.1M chloride). In a preferred embodiment, the potential is stepped first to a positive applied potential of 0.15V, for a period of about 1 to 4 seconds, and then stepped to a negative applied potential when it is desired to measure the reduction current. Where a different redox agent is used, the applied potentials can be varied in accordance with the potentials at which the oxidation/reduction peak occurs. The length of time the potential is applied may also vary.

The electrochemical test of the invention therefore enables a measurement of cholesterol and/or triglyceride to be made in a very short period of time, typically within about five minutes or within four minutes from application of a sample to the device.

The invention also provides the use of a surfactant of the formula (1) (preferably a surfactant of formula (I)) for breaking down all lipoprotein fractions in a sample to determine the total amount of cholesterol and/or triglyceride in the sample.

In yet another specific embodiment, the invention provides a sensor for determining the amount of triglyceride and/or cholesterol in a sample, the sensor comprising

(a) a surfactant of formula (Ix) or (II)

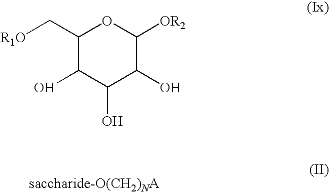

wherein R₁ is a group of formula —CONH(CH₂)_{M}—CH₃ wherein M is from 6 to 20, R₂ is hydrogen or methyl, A is a C₃-C₈ cylcoalkyl group and N is from 1 to 10; and

(b) an enzyme reagent for measuring triglycerides and/or an enzyme reagent for measuring cholesterol.

In this embodiment, the surfactant is preferably of formula (Ixx):

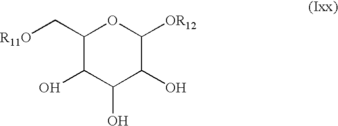

wherein either R₁₁ is a group of formula —CONH(CH₂)_{M}—CH₃ and R₁₂ is hydrogen or methyl; or R₁₁ is hydrogen and R₁₂ is —(CH₂)_{N}-A. It is preferred that M is from 6 to 10, N is from 2 to 6 and A is cyclohexyl. Specific preferred surfactants are methyl-6-O—(N-heptylcarbamoyl)-D-glucopyranoside or 3-cyclohexyl-1-propyl-D-glucoside.

In this embodiment, the sensor may additionally comprise an electrochemical cell having at least two electrodes; a coenzyme and a redox agent capable of being oxidized or reduced to form a product; and optionally additionally a reductase.

Typically, the surfactant, enzyme reagent and, if used, the coenzyme, redox agent and/or reductase are present as a single reagent mixture. The enzyme reagent for measuring cholesterol may, for example, comprise (i) cholesterol esterase or a lipase and (ii) cholesterol dehydrogenase. The enzyme reagent for measuring triglyceride may, for example, comprise (i) cholesterol esterase or a lipase and (ii) glycerol dehydrogenase. For example, the enzyme reagent for measuring triglyceride may comprise a lipase and glycerol dehydrogenase.

Also provided in this specific embodiment is a method for determining the amount of cholesterol and/or triglyceride in a sample, the method comprising:
contacting the sample with a surfactant of this embodiment; and
determining the amount of cholesterol and/or triglyceride present.

In this method the determination of the amount of cholesterol and/or triglyceride is typically an electrochemical determination. For example, the method may comprise contacting the sample with the surfactant, an enzyme reagent, a coenzyme, a redox agent and optionally a reductase, in an electrochemical cell, applying a potential across the electrochemical cell and determining the electrochemical response of the cell. Preferably, the electrochemical response of the cell is determined at least 1.5 minutes after contacting the sample with the surfactant. Preferably, the amount of surfactant which is contacted with the sample is sufficient to provide a surfactant concentration of at least 20 mM in the combination of sample and surfactant.

### Example 1

### Anameg-7 and Cyglu-3

The aim of the experiment was to investigate cholesterol sensors prepared with novel surfactants for their effect on measurement of plasma HDL or LDL.

Methods

30 mM Ru(Acac) Solution

Ru(Acac) solution was prepared by mixing Tris buffer, KOH, β-Lactose and Ru(Acac) to provide a solution containing 100 mM Tris buffer pH 9.0, 30 mM KOH, 10% w/v β-Lactose and 30 mM Ru(Acac). This solution was mixed using a Covaris acoustic mixer. Ru(Acac)=Ru(acac)₂(Py-3-CO2H)(Py-3-CO2)

Anameg-7 and Cyglu-3 Solutions

A double strength Anameg-7 or Cyglu-3 solution was made by addition of the relevant surfactant to Ru(Acac) solution to provide the following final concentrations:

Anameg-7 (Anatrace, A340)

200 mM (0.0088 g in 131 μl Ru(Acac) solution)

100 mM (37.5 μl of 200 mM stock+37.5 μl Ru(Acac) solution)

50 mM (24 μl of 200 mM stock+75 μl Ru(Acac) solution)

Cyglu-3 (Anatrace, C323G)

200 mM (0.0077 g in 125 μl Ru(Acac) solution)

100 mM (37.5 μl of 200 mM stock+37.5 μl Ru(Acac) solution)

50 mM (25 μl of 200 mM stock+75 μl Ru(Acac) solution)

Enzyme Mixture

Enzyme mixture was made at double strength by addition of enzymes and cofactor to Ru(Acac) solution to produce the following final concentrations:

17.7 mM Thionicotinamide adenine dinucleotide (Oriental Yeast Co)

8.4 mg/ml Putidaredoxin Reductase (Biocatalysts)

6.7 mg/ml Lipase (Genzyme)

44.4 mg/ml Cholesterol Dehydrogenase, Gelatin free (Amano, CHDH-6)

This solution was mixed using a Covaris acoustic mixer.

Dispense and Freeze Drying

For each enzyme solution, equal volumes (approximately 50 μl) of double concentration enzyme solution and Anameg-7 or Cyglu-3 solutions were mixed 1:1 to give the final enzyme/surfactant mixes. 0.4 ml/well of each solution was dispensed onto sensors as described in WO 2006/000828 using an electronic pipette. The dispensed sensor sheets were then freeze dried.

Plasma Samples

Plasma samples were defrosted for 30 minutes before being centrifuged for 5 minutes at 2900 RCF. Delipidated serum (Scipac, S139) was also used as a sample. The samples were analysed using a Space clinical analyser (Schiappanelli Biosystems Inc) for total cholesterol, triglyceride (TG), HDL cholesterol and LDL cholesterol concentrations.

Testing Protocol 1

15 μl of a plasma sample was used per electrochemical cell. On the addition of 15 μl of plasma a chronoamperometry test was initiated. The oxidation current is measured at 0.15 V at 13 time points (0, 32, 64, 96, 128, 160, 192, 224, 256, 288, 320, 352 and 384 seconds), with a reduction current measured at −0.45 V at the final time point (416 seconds). The current was measured for 4 seconds at each of the specified time points. Each sample was tested in duplicate.

Testing Protocol 2

15 μl of a plasma sample was used per electrochemical cell. On the addition of 1.5 μl of plasma a chronoamperometry test was initiated. The oxidation current is measured at 0.15 V at 13 time points (0, 34, 68, 102, 136, 170, 204, 238, 272, 306, 340, 374 and 408 seconds), with a reduction current measured at −0.45 V at the final time point (442 seconds). The current was measured for 4 seconds at each of the specified time points. Each sample was tested in duplicate.

Analysis

The current measurements collected were plotted against the HDL and LDL'cholesterol concentrations of the plasma samples as measured by the space analyser. The gradient for each time point was used to calculate the % differentiation obtained between measurement of LDL and HDL. The slopes and intercepts for the calibration plots to HDL and LDL at each time point are given in Table 1A and show that these surfactants act on both HDL and LDL particularly at times in excess of 32 seconds.

The differentiation between HDL and LDL can be determined for HDL according to the equation (i):

$$Differentiation ( \% ) = \frac{{G}_{HDL} {\_G}_{LDL}}{{G}_{HDL}} \times 100 \qquad ( i )$$

wherein Gₓ is the gradient of the measured response to X (e.g. the measured current vs. the known concentration of X). The measured response may be any measured value which relates (or corresponds) to the lipoprotein concentration, for example which is proportional to the lipoprotein concentration.

For sensors prepared with Anameg-7, the HDL gradient of response is low and decreases with increasing surfactant concentration. The LDL gradient of response increases with surfactant concentration, and is relatively high. This may indicate that HDL has reacted with the surfactant very quickly, followed by a strong response to LDL. A similar effect is seen with sensors prepared with Cyglu-3.

The sensor responses were also plotted vs. the total cholesterol concentrations as measured by the Space analyser. The slopes and intercepts for the calibration plots to total cholesterol at each time point are given in Table 1B. At long times, the gradients of response to total cholesterol are high for 100 mM Anameg-7 or Cyglu-3, and the intercepts are low, indicating the sensors respond to total cholesterol rather than HDL or LDL cholesterol.

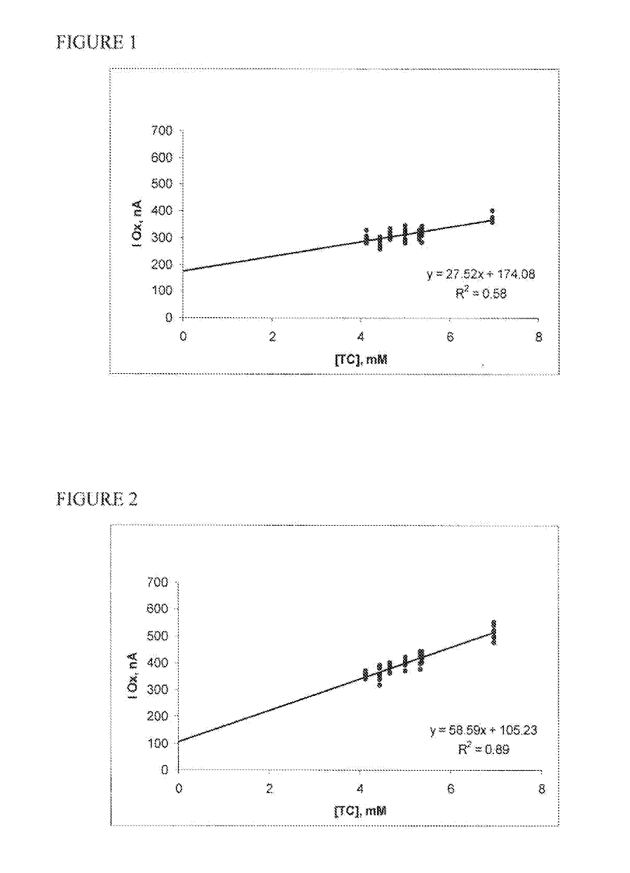

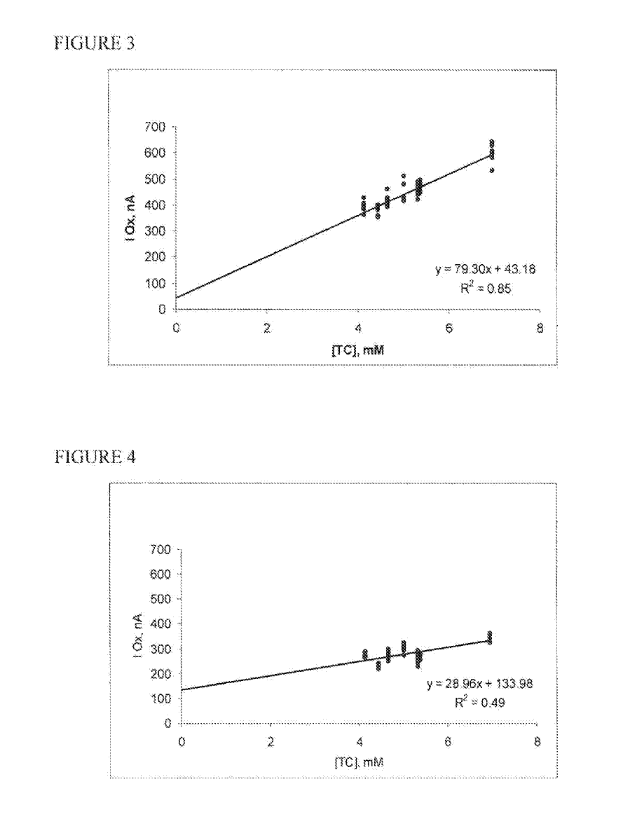

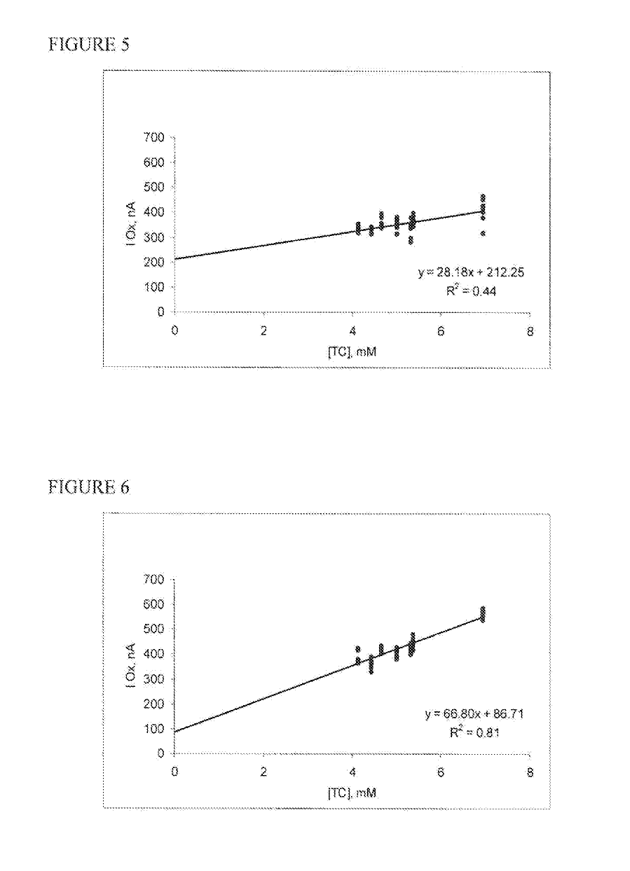

Calibration plots for response to total cholesterol are shown in the Figures. FIGS. 1 to 3 relate to the current measurements taken at 288 seconds using Anameg-7 as the surfactant ((a) 25 mM Anameg-7; (b) 50 mM Anameg-7; (c) 100 mM Anameg-7) and FIGS. 4 to 6 relate to current measurements taken at 320 seconds using Cyglu-3 as the surfactant ((d) 25 mM Cyglu-3; (e) 50 mM Cyglu-3; (f) 100 mM Cyglu-3).

FIGS. 1-6 show that sensors containing 100 mM Anameg-7 or Cyglu-3 give good linearity and high gradients of response to total cholesterol.

### Examples 2 to 14

Several experiments were performed using the same basic enzyme mix, with small changes in the generic formulation and/or test procedure. The modifications described in each of these Examples relate to changes made to the generic details set out below.

Enzyme Mixture

The generic enzyme mixture contained the following constituents:

0.1M Tris buffer (pH 9.0).

40 mM KOH

40 mM Ru(Acac)

10% lactose

100 mM Anameg 7

500 mM NaCl

8.9 mM thionicotinamide adenine dinucleotide (TNAD)

4.2 mg/ml Putidaredoxin Reductase (PdR)

3.3 mg/ml lipase

22 mg/ml Cholesterol Dehydrogenase, gelatin free (ChDH).

Dispense and Freeze Drying

0.4 μl/well of each solution was dispensed onto sensors as described in WO 2006/000828 using an electronic pipette. The dispensed sensor sheets were then freeze dried.

Plasma Samples

Sensors were tested with plasma or delipidated serum (Scipac, S139). The samples were analysed using a Space clinical analyser (Schiappanelli Biosystems Inc.) for both total cholesterol and triglyceride concentrations.

Testing Protocol

12 μl of plasma sample were added to each electrochemical cell. On addition of the sample a chronoamperometry test was initiated and the same series of current measurements obtained as in Protocol 1 in Example 1.

The sensor responses were plotted against the total cholesterol concentrations as measured by the Space analyser. The slopes and intercepts for the calibration plots to total cholesterol at selected time points were then calculated.

### Example 2

### Different Alkyl Chain Lengths

The formulation and test procedure were as set out above, with the following modifications:

30 mM KOH was used rather than 40 mM KOH

30 mM Ru(Acac) was used rather than 40 mM Ru(Acac)

No NaCl was present in the mixture.

The Anameg-7 surfactant was replaced by the following surfactants, which were used at concentrations of 0, 25, 50 and 100 mM:
n-hexyl-β-D-glucopyranoside (HexGP, available from Anatrace)
n-heptyl-β-D-glucopyranoside (HepGP, available from Anatrace)
n-nonyl-β-D-glucopyranoside (OGP, available from Anatrace)
n-nonyl-β-D-glucopyranoside (NGP, available from Anatrace)

The results are presented in Table 2.

### Example 3

### Different Ionic Strengths

The formulation and test procedure were as set out above, with the following modifications:

Salts were added or removed from the generic enzyme mixture as follows:
0 mM salt present in mixture
250 mM KCl or NaCl present in mixture
500 mM KCl present in mixture

The results are presented in Table 3.

### Example 4

### Replacement of Lactose with BSA

The formulation and test procedure were as set out above, but lactose was replaced by BSA at concentrations of 1%, 2% and 3%.

The results are presented in Table 4.

### Example 5

### Variation in Cholesterol Dehydrogenase Concentration

The formulation and test procedure were as set out above, but the cholesterol dehydrogenase concentration was tested at 22, 44, and 66 mg/ml.

Testing was completed according to Protocol 2 in Example 1.

The results are presented in Table 5.

### Example 6

### Replacement of Iris Buffer with Diethanolamine Buffer

The formulation and test procedure were as set out above, but the buffer was changed from Tris to 0.1 M diethanolamine (pH 8.6).

The following additional modifications were also made:

10% lactose was replaced by the combination of 1% w/v myo-inositol and 1% w/v ectoine.

500 mM NaCl was replaced by 400 mM KCl

40 mM Ru(Acac) was replaced by 80 mM Ruthenium hexamine (Ru(NH₃)₆Cl₃).

Furthermore, the 100 mM Anameg 7 of the generic enzyme mixture was replaced by the following surfactants:
200 mM Anameg-7
100 or 200 mM Cyglu-3
100 or 200 mM octylglucopyranoside (OGP)
100 or 200 mM n-nonyl-β-D-glucopyranoside (NGP)
5% w/v CHAPS:5% w/v DeoxyBigCHAPS (Reference Example)

In this Example, the testing protocol was modified slightly, as follows. The oxidation current was measured at +0.15 V for 1.0 second at 15 consecutive time intervals, for a period of 196 seconds, followed by measuring a reduction current at −0.45 V for 1.0 second. oxidations were at approximately 0, 14, 28, 42, 56, 70, 84, 98, 112, 126, 140, 154, 168, 182, 196 seconds, with the reduction current at −0.45 V measured at 210 seconds.

The results are presented in Table 6.

### Example 7

### Varying Surfactant Concentration Over a Wide Range

The formulation and test procedure were as set out above, but the surfactant used and its concentration were varied as follows (in place of the 100 mM Anameg 7 in the generic mixture):
No surfactant
10, 25, 50, 200 or 300 mM Anameg-7
10, 25, 50, 100, 200 or 300 mM Cyglu-3
10, 25, 50, 100, 200 or 300 mM n-nonyl-β-D-glucopyranoside (NGP)

The testing protocol was as in Protocol 2 in Example 1.

The results are presented in Tables 7A and 7B.

### Example 8

### Cholate-Free Cholesterol Dehydrogenase

The formulation and test procedure were as set out above, but the standard cholesterol dehydrogenase was replaced by cholate-free dehydrogenase.

The testing protocol was as set out in Example 7.

The results are presented in Table 8.

### Example 9

### Dual Surfactant Systems

The formulation and test procedure were as set out above, but the 100 mM Anameg of the generic enzyme mix was replaced by the following combinations of surfactants:
50 mM Anameg-7 and 50 mM n-nonyl-β-D-glucopyranoside (NGP)
50 mM Cyglu-3 and 50 mM n-nonyl-β-D-glucopyranoside (NGP)

The testing protocol was as set out in Example 7.

The results are presented in Table 9.

### Example 10

### Use of a Different, Novel, Mediator

The formulation and test procedure were as set out above, but the 40 mM Ru(Acac) present in the generic enzyme mixture was replaced by a different ruthenium mediator.

The experiment was carried using the 100 mM Anameg-7 surfactant component present in the generic enzyme mixture. Further experiments were then carried out using the following alternative surfactants:
No surfactant
100 mM Cyglu-3
100 mM n-nonyl-β-D-glucopyranoside (NGP)

The testing protocol was as set out in Example 7.

The results are presented in Table 10.

### Example 11

### Different Sugar Surfactants

The formulation and test procedure were as set out above, but the 100 mM Anameg-7 of the generic enzyme mixture was replaced as follows:
No surfactant
50 mM n-octyl β-D-galactopyranoside
50 mM n-heptyl β-D-thioglucopyranoside
50 mM N-octanoyl β-D-glucosylamine (NOGA)
50 mM Anameg-7
50 mM n-octyl β-D-glucopyranoside (OGP)
50 mM n-heptyl β-D-glucopyranoside (HeptGP)
100 mM NOGA
50, 100 or 200 mM n-octyl β-D-glucopyranoside (OGP)
50 or 100 mM n-octyl β-D-mannopyranoside (OMP)
200 mM n-octyl β-D-mannopyranoside (OMP)
50, 100 or 200 mM n-octanoyl D-glucopyranoside (OYGP)

The testing protocol was as set out in Example 7.

The results are presented in Table 11.

### Example 12

### Range of Anameg Surfactants

The formulation and test procedure were as set out above, but a range of Anameg surfactants were run as follows:
No surfactant
50 mM, 100 mM or 200 mM Anameg-5
50 mM, 100 mM or 200 mM Anameg-6
50 mM, 100 mM or 200 mM Anameg-7
50 mM, 100 mM or 200 mM Anameg-8
50 mM, 100 mM or 200 mM Anameg-9

The testing protocol was as set out in Example 7.

Results are presented in Table 12.

### Example 13

### Range of Cyglu Surfactants

The formulation and test procedure were as set out above, but a range of Cyglu surfactants replaced the Anameg-7 as follows:
No surfactant
0, 50, 100 or 200 mM β-cyglu-1
50, 100 or 200 mM β-cyglu-2
50, 100 or 200 mM β-cyglu-3
50, 100 or 200 mM α-cyglu-3

The testing protocol was as set out in Example 7.

Results are presented in Table 13.

### Example 14

### Replacing Enzymes with Comparable Ones

The formulation and test procedure were as set out above, but either a different NADH oxidase or a different ester cleaving enzyme was used as follows:
4.2 mg/ml Putidaredoxin Reductase was replaced by 4.2 mg/mL diaphorase
3.3 mg/ml Lipase (Genzyme) was replaced by 3.3 mg/mL Toyobo ChE

Additionally, 10% lactose was replaced with 2% w/v BSA

The testing protocol was as set out in Example 7.

Results are presented in Table 14.

### Example 15

### Freeze-Dried Triglyceride Sensors

In this Example, the final enzyme mix for the triglyceride sensor contained:

0.1M HEPBS (pH 9.0)

30 mM KOH

30 mM Ru(Acac)

10% w/v lactose

17.6 mM Thionicotinamide adenine dinucleotide (TNAD)

6.7 mg/ml diaphorase

5 mg/ml Toyobo ChE

45 mg/ml Glycerol Dehydrogenase

This formulation was used both without surfactant and with the inclusion of the following surfactants at the following concentrations:
1% and 5% w/v Anameg-7
1% and 5% w/v cyglu-3
1% and 5% w/v n-nonyl-β-D-glucopyranoside (NGP)

Dispense and Freeze Drying

0.3 μl/well of each solution was dispensed onto sensors as described in WO 2006/000828 using an electronic pipette. The dispensed sensor sheets were then freeze dried.

Plasma Samples

Sensors were tested with plasma or delipidated serum (Scipac, S139). The samples were analysed using a Space clinical analyser (Schiappanelli Biosystems Inc.) for both total cholesterol and triglyceride concentrations.

Testing Protocol

12 μl of plasma sample were added to each electrochemical cell the rest of the testing protocol was as set out in Example 7.

The sensor responses were plotted against the total triglyceride concentrations as measured by the Space analyser. The slopes and intercepts for the calibration plots to triglycerides at selected time points were then calculated.

The results are presented in Table 15.

TABLE 1A

| | | time | | | | | | | | | | | | |
|---|---|---|---|---|---|---|---|---|---|---|---|---|---|---|
| | | 0 | 32 | 64 | 96 | 128 | 160 | 192 | 224 | 256 | 288 | 320 | 352 | 384 |
| 25 mM | | | | | | | | | | | | | | |
| anameg-7 | | | | | | | | | | | | | | |
| LDL | slope | 2.83 | 6.09 | 8.92 | 11.01 | 13.45 | 16.44 | 19.43 | 21.48 | 23.13 | 24.31 | 25.88 | 26.21 | 26.85 |
| | intercept | 144.68 | 190.99 | 210.17 | 220.56 | 228.06 | 233.01 | 236.51 | 240.65 | 243.96 | 247.37 | 249.28 | 254.40 | 258.20 |
| HDL | slope | 20.63 | 21.50 | 20.94 | 21.07 | 19.77 | 18.44 | 17.18 | 15.67 | 12.83 | 9.84 | 7.52 | 5.90 | 4.54 |
| | intercept | 113.72 | 170.43 | 197.51 | 213.12 | 229.85 | 245.57 | 259.48 | 271.70 | 284.84 | 297.28 | 308.03 | 316.82 | 324.93 |
| | % diff | 86.27 | 71.69 | 57.40 | 47.77 | 31.98 | 10.85 | −11.58 | −27.02 | −44.54 | −59.51 | −70.93 | −77.51 | −83.07 |
| 50 mM | | | | | | | | | | | | | | |
| anameg-7 | | | | | | | | | | | | | | |
| LDL | slope | 12.75 | 14.68 | 24.34 | 31.51 | 36.68 | 41.78 | 46.32 | 49.26 | 51.24 | 52.43 | 53.22 | 53.72 | 54.31 |
| | intercept | 146.41 | 220.57 | 235.09 | 240.18 | 244.46 | 245.71 | 245.35 | 246.76 | 248.58 | 251.03 | 253.67 | 255.86 | 256.79 |
| HDL | slope | 14.74 | 21.67 | 16.73 | 10.88 | 4.83 | 0.62 | −2.73 | −4.25 | −5.51 | −6.78 | −7.44 | −7.55 | −7.85 |
| | intercept | 159.27 | 229.24 | 280.16 | 316.17 | 346.18 | 369.46 | 388.08 | 400.42 | 409.96 | 417.75 | 423.38 | 426.98 | 429.98 |
| | % diff | 13.47 | 32.29 | −31.28 | −65.46 | −86.84 | −98.53 | −105.90 | −108.62 | −110.74 | −112.93 | −113.97 | −114.05 | −114.45 |
| 100 mM | | | | | | | | | | | | | | |
| anameg-7 | | | | | | | | | | | | | | |
| LDL | slope | 16.49 | 33.75 | 54.02 | 59.57 | 60.94 | 62.57 | 64.85 | 67.59 | 68.87 | 68.93 | 68.49 | 68.62 | 68.92 |
| | intercept | 161.58 | 217.18 | 202.02 | 219.40 | 237.05 | 244.61 | 246.73 | 247.08 | 249.68 | 253.62 | 257.92 | 259.72 | 260.79 |
| HDL | slope | 8.20 | 10.93 | −0.50 | −5.75 | −5.33 | −3.39 | −0.94 | 0.82 | 1.37 | 1.83 | 2.65 | 2.74 | 2.41 |
| | intercept | 205.51 | 303.27 | 374.25 | 410.41 | 426.29 | 433.55 | 437.05 | 440.75 | 445.00 | 447.74 | 448.44 | 449.80 | 451.56 |
| | % diff | −50.27 | −67.62 | −100.93 | −109.66 | −108.75 | −105.42 | −101.45 | −98.79 | −98.02 | −97.35 | −96.14 | −96.00 | −96.51 |
| 25 mM | | | | | | | | | | | | | | |
| cyglu-3 | | | | | | | | | | | | | | |
| LDL | slope | −0.61 | 7.08 | 10.49 | 12.51 | 14.55 | 17.14 | 19.02 | 20.68 | 22.24 | 23.91 | 25.96 | 27.60 | 28.60 |
| | intercept | 111.78 | 152.49 | 169.57 | 180.37 | 189.48 | 194.63 | 200.67 | 205.66 | 209.15 | 211.48 | 211.25 | 211.73 | 214.59 |
| HDL | slope | 27.63 | 37.01 | 38.21 | 39.13 | 40.18 | 40.57 | 40.27 | 38.92 | 35.74 | 32.63 | 29.49 | 26.17 | 23.18 |
| | intercept | 61.99 | 109.68 | 134.21 | 149.01 | 161.25 | 172.58 | 183.52 | 194.76 | 207.74 | 219.92 | 231.10 | 241.91 | 251.74 |
| | % diff | 102.23 | 80.86 | 72.55 | 68.04 | 63.80 | 57.76 | 52.78 | 46.87 | 37.79 | 26.73 | 11.98 | −5.21 | −18.93 |
| 50 mM | | | | | | | | | | | | | | |
| cyglu-3 | | | | | | | | | | | | | | |
| LDL | slope | 2.85 | 15.78 | 18.62 | 19.97 | 20.58 | 20.91 | 21.93 | 22.86 | 24.37 | 25.30 | 27.04 | 28.95 | 30.63 |
| | intercept | 124.98 | 164.63 | 194.07 | 212.65 | 230.19 | 247.11 | 259.12 | 268.21 | 272.89 | 277.47 | 278.49 | 277.36 | 275.88 |
| HDL | slope | 24.67 | 22.79 | 20.38 | 17.91 | 15.96 | 14.86 | 14.82 | 14.28 | 13.23 | 12.47 | 10.85 | 8.75 | 6.08 |
| | intercept | 91.60 | 174.41 | 214.85 | 240.97 | 262.93 | 281.72 | 296.43 | 308.70 | 319.93 | 328.61 | 338.02 | 346.77 | 355.28 |
| | % diff | 88.44 | 30.78 | 8.60 | −10.31 | −22.48 | −28.93 | −32.42 | −37.52 | −45.69 | −50.72 | −59.87 | −69.78 | −80.16 |
| 100 mM | | | | | | | | | | | | | | |
| cyglu-3 | | | | | | | | | | | | | | |
| LDL | slope | 6.60 | 25.37 | 34.46 | 41.21 | 46.20 | 50.40 | 53.97 | 57.31 | 59.23 | 59.50 | 58.62 | 57.45 | 56.02 |
| | intercept | 171.29 | 203.11 | 218.60 | 229.23 | 241.09 | 251.01 | 255.41 | 255.92 | 258.14 | 262.85 | 268.65 | 274.28 | 280.37 |
| HDL | slope | 23.68 | 7.40 | 1.60 | −1.08 | −4.07 | −5.76 | −8.31 | −7.65 | −6.74 | −5.67 | −5.42 | −3.50 | −2.39 |
| | intercept | 143.01 | 264.09 | 315.80 | 350.35 | 380.76 | 403.89 | 423.56 | 431.93 | 436.42 | 438.62 | 440.81 | 438.97 | 438.05 |
| | % diff | 72.13 | −70.83 | −95.36 | −102.63 | −108.81 | −111.42 | −115.40 | −113.35 | −111.39 | −109.53 | −109.25 | −106.08 | −104.26 |

TABLE 1B

| | | Time in seconds | | | | | | | | | | | | |
|---|---|---|---|---|---|---|---|---|---|---|---|---|---|---|
| | | 0 | 32 | 64 | 96 | 128 | 160 | 192 | 224 | 256 | 288 | 320 | 352 | 384 |
| 25 mM | | | | | | | | | | | | | | |
| Anameg 7 | | | | | | | | | | | | | | |
| TC | Slope | 4 | 7 | 11 | 13 | 15 | 18 | 22 | 25 | 26 | 28 | 29 | 30 | 30 |
| | Intercept | 130 | 173 | 180 | 185 | 186 | 184 | 178 | 174 | 173 | 174 | 172 | 176 | 179 |
| 50 mM | | | | | | | | | | | | | | |
| Anameg 7 | | | | | | | | | | | | | | |
| TC | Slope | 15 | 16 | 26 | 34 | 40 | 46 | 51 | 55 | 57 | 59 | 60 | 60 | 61 |
| | Intercept | 110 | 186 | 175 | 161 | 150 | 136 | 121 | 112 | 107 | 105 | 104 | 104 | 103 |
| 100 mM | | | | | | | | | | | | | | |
| Anameg 7 | | | | | | | | | | | | | | |
| TC | Slope | 13 | 33 | 52 | 62 | 66 | 69 | 73 | 77 | 79 | 79 | 80 | 80 | 81 |
| | Intercept | 152 | 154 | 104 | 83 | 78 | 71 | 62 | 49 | 43 | 43 | 44 | 43 | 40 |
| 25 mM | | | | | | | | | | | | | | |
| Cyglu 3 | | | | | | | | | | | | | | |
| TC | Slope | 1 | 7 | 11 | 13 | 15 | 18 | 21 | 23 | 25 | 27 | 29 | 31 | 32 |
| | Intercept | 107 | 136 | 146 | 152 | 154 | 151 | 148 | 146 | 143 | 139 | 134 | 129 | 127 |
| 50 mM | | | | | | | | | | | | | | |
| Cyglu 3 | | | | | | | | | | | | | | |
| TC | Slope | 2 | 14 | 18 | 19 | 20 | 20 | 21 | 23 | 25 | 26 | 28 | 31 | 33 |
| | Intercept | 126 | 142 | 160 | 175 | 190 | 204 | 212 | 216 | 217 | 216 | 212 | 205 | 198 |
| 100 mM | | | | | | | | | | | | | | |
| Cyglu 3 | | | | | | | | | | | | | | |
| TC | Slope | 7 | 25 | 34 | 41 | 47 | 53 | 57 | 62 | 65 | 67 | 67 | 66 | 66 |
| | Intercept | 149 | 147 | 142 | 135 | 130 | 122 | 114 | 99 | 88 | 86 | 87 | 91 | 95 |

TABLE 2

| | | Time | | |
|---|---|---|---|---|
| | | 0 | 192 | 384 |
| Blank | Slope | 9.0 | 21.3 | 25.5 |
| | Intercept | 49.9 | 85.7 | 104.9 |
| 25 mM | Slope | 11.6 | 23.1 | 24.1 |
| HexGP | Intercept | 40.0 | 86.6 | 120.8 |
| 50 mM | Slope | 17.4 | 31.3 | 33.6 |
| HexGP | Intercept | 21.6 | 63.4 | 86.5 |
| 100 mM | Slope | 8.3 | 23.6 | 28.7 |
| HexGP | Intercept | 63.7 | 109.0 | 126.5 |
| 25 mM | Slope | 7.6 | 17.5 | 19.6 |
| HeptGP | Intercept | 72.2 | 114.7 | 126.1 |
| 50 mM | Slope | 3.4 | 14.1 | 16.0 |
| HeptGP | Intercept | 92.8 | 142.4 | 152.2 |
| 100 mM | Slope | 9.8 | 22.2 | 28.0 |
| HeptGP | Intercept | 55.8 | 127.7 | 119.9 |
| 25 mM | Slope | 10.5 | 16.7 | 24.8 |
| OGP | Intercept | 46.8 | 125.9 | 118.0 |
| 50 mM | Slope | 12.9 | 24.5 | 30.1 |
| OGP | Intercept | 60.3 | 119.0 | 114.8 |
| 100 mM | Slope | 15.7 | 38.2 | 57.3 |
| OGP | Intercept | 85.0 | 156.0 | 95.6 |
| 25 mM | Slope | 10.5 | 38.1 | 47.0 |
| NGP | Intercept | 85.4 | 140.3 | 154.4 |
| 50 mM | Slope | 15.6 | 58.2 | 68.3 |
| NGP | Intercept | 114.2 | 151.9 | 148.8 |
| 100 mM | Slope | 20.2 | 83.2 | 101.1 |
| NGP | Intercept | 125.5 | 77.8 | 35.4 |

TABLE 3

| | | Time | | |
|---|---|---|---|---|
| | | 0 | 192 | 384 |
| Blank | Slope | 18.7 | 88.6 | 113.4 |
| | Intercept | 122.8 | 29.6 | −14.6 |
| 250 mM | Slope | 9.5 | 85.9 | 93.6 |
| NaCl | Intercept | 124.7 | 40.7 | 20.8 |
| 500 mM | Slope | 21.7 | 82.8 | 82.7 |
| NaCl | Intercept | 138.2 | 63.3 | 65.6 |
| 250 mM | Slope | 22.6 | 79.3 | 82.4 |
| KCl | Intercept | 127.4 | 79.9 | 75.7 |
| 500 mM | Slope | 26.0 | 84.1 | 88.8 |
| KCl | Intercept | 136.6 | 86.8 | 67.2 |

TABLE 4

| | | Time | | |
|---|---|---|---|---|
| | | 0 | 160 | 384 |
| 1% BSA | Slope | 25.2 | 47.1 | 61.9 |
| | Intercept | 52.1 | 152.9 | 135.8 |
| 2% BSA | Slope | 18.6 | 52.4 | 58.8 |
| | Intercept | 93.1 | 144.2 | 162.8 |
| 3% BSA | Slope | 15.0 | 39.1 | 64.2 |
| | Intercept | 96.1 | 171.6 | 118.0 |

TABLE 5

| | | Time | | |
|---|---|---|---|---|
| | | 0 | 160 | 384 |
| 22 mg/ml | Slope | 26.2 | 70.8 | 84.4 |
| ChDH | Intercept | 96.2 | 98.1 | 50.8 |
| 44 mg/ml | Slope | 22.8 | 70.0 | 78.4 |
| ChDH | Intercept | 104.4 | 73.7 | 37.7 |
| 66 mg/ml | Slope | 14.7 | 65.9 | 72.1 |
| ChDH | Intercept | 116.1 | 79.2 | 56.5 |

TABLE 6

| | | Time | | |
|---|---|---|---|---|
| | | 0 | 98 | 196 |
| 200 mM | Slope | −19.6 | 98.0 | 163.7 |
| Anameg-7 | Intercept | 366.8 | 465.8 | 167.3 |
| 100 mM | Slope | 18.0 | 95.8 | 130.0 |
| Anameg-7 | Intercept | 154.8 | 354.0 | 267.1 |
| 200 mM cyglu-3 | Slope | 26.3 | 93.0 | 127.4 |
| | Intercept | 150.2 | 481.8 | 302.0 |
| 100 mM cyglu-3 | Slope | 17.3 | 64.1 | 88.6 |
| | Intercept | 129.3 | 368.7 | 348.6 |
| 200 mM OGP | Slope | 12.8 | 69.7 | 96.7 |
| | Intercept | 261.4 | 457.2 | 364.6 |
| 100 mM OGP | Slope | 20.5 | 64.3 | 85.2 |
| | Intercept | 181.2 | 356.6 | 361.4 |
| 200 mM NGP | Slope | 18.3 | 92.2 | 121.5 |
| | Intercept | 169.6 | 252.1 | 146.0 |
| 100 mM NGP | Slope | −0.2 | 58.0 | 89.3 |
| | Intercept | 285.6 | 394.5 | 309.1 |
| 5% w/v | Slope | 12.8 | 124.7 | 170.6 |
| CHAPS, 5% | Intercept | 231.5 | 254.1 | 3.2 |
| w/v deoxy | | | | |
| bigCHAP | | | | |

TABLE 7A

| | | Time | | |
|---|---|---|---|---|
| | | 0 | 204 | 408 |
| 0 mM | Slope | 2.1 | 9.0 | 14.2 |
| Anameg-7 | Intercept | 68.4 | 146.5 | 153.1 |
| 10 mM | Slope | 2.5 | 12.9 | 19.9 |
| Anameg-7 | Intercept | 93.2 | 132.8 | 132.2 |
| 25 mM | Slope | 4.1 | 12.3 | 20.5 |
| Anameg-7 | Intercept | 104.4 | 150.9 | 155.6 |
| 50 mM | Slope | 8.6 | 26.9 | 36.7 |
| Anameg-7 | Intercept | 112.4 | 153.2 | 155.2 |
| 100 mM | Slope | 15.2 | 60.3 | 63.2 |
| Anameg-7 | Intercept | 123.8 | 113.5 | 119.1 |
| 200 mM | Slope | 19.9 | 68.0 | 71.0 |
| Anameg-7 | Intercept | 95.8 | 45.1 | 38.6 |
| 300 mM | Slope | 15.6 | 64.7 | 72.1 |
| Anameg-7 | Intercept | 70.1 | 16.7 | 19.2 |

TABLE 7B

| | | Time | | |
|---|---|---|---|---|
| | | 0 | 204 | 408 |
| Blank | Slope | −0.3 | 7.3 | 15.0 |
| (Cyglu- | Intercept | 78.3 | 152.6 | 146.7 |
| 3 or | | | | |
| NGP) | | | | |
| 10 mM | Slope | 3.0 | 9.2 | 16.4 |
| Cyglu-3 | Intercept | 86.4 | 152.7 | 158.9 |
| 25 mM | Slope | 3.2 | 15.6 | 24.4 |
| Cyglu-3 | Intercept | 108.9 | 164.9 | 174.4 |
| 50 mM | Slope | 7.3 | 19.3 | 25.3 |
| Cyglu-3 | Intercept | 85.9 | 167.2 | 179.8 |
| 100 mM | Slope | 16.6 | 51.3 | 57.5 |
| Cyglu-3 | Intercept | 100.3 | 139.5 | 133.0 |
| 200 mM | Slope | 19.4 | 39.4 | 45.8 |
| Cyglu-3 | Intercept | 60.7 | 101.4 | 100.4 |
| 300 mM | Slope | 10.9 | 36.1 | 46.1 |
| Cyglu-3 | Intercept | 74.4 | 85.4 | 71.5 |
| 10 mM | Slope | 2.7 | 13.3 | 18.8 |
| NGP | Intercept | 109.7 | 142.7 | 157.1 |
| 25 mM | Slope | 13.4 | 27.4 | 37.2 |
| NGP | Intercept | 106.5 | 170.7 | 160.0 |
| 50 mM | Slope | 16.5 | 37.9 | 45.7 |
| NGP | Intercept | 96.8 | 160.6 | 153.8 |
| 100 mM | Slope | 21.5 | 54.7 | 55.0 |
| NGP | Intercept | 115.2 | 140.9 | 164.3 |
| 200 mM | Slope | 20.4 | 59.0 | 59.4 |
| NGP | Intercept | 97.4 | 101.4 | 111.4 |
| 300 mM | Slope | 16.2 | 54.5 | 50.6 |
| NGP | Intercept | 74.4 | 52.2 | 88.2 |

TABLE 8

| | | Time | | |
|---|---|---|---|---|
| | | 0 | 204 | 408 |
| cholate free ChDH, | Slope | 19.6 | 62.0 | 67.4 |
| 100 mM | Intercept | 103.2 | 120.1 | 118.6 |
| Anameg-7 | | | | |
| standard ChDH, | Slope | 29.7 | 65.5 | 70.4 |
| 100 mM Anameg-7 | Intercept | 74.6 | 100.3 | 99.0 |
| cholate free ChDH, | Slope | 18.5 | 45.9 | 54.8 |
| 100 mM Cyglu-3 | Intercept | 59.4 | 164.1 | 161.8 |
| standard ChDH, | Slope | 19.4 | 52.9 | 57.4 |
| 100 mM Cyglu-3 | Intercept | 58.0 | 108.4 | 110.5 |
| cholate free ChDH, | Slope | 29.7 | 65.5 | 70.4 |
| 100 mM NGP | Intercept | 74.6 | 100.3 | 99.0 |
| standard ChDH, | Slope | 13.3 | 59.2 | 65.6 |
| 100 mM NGP | Intercept | 116.6 | 135.6 | 131.5 |

TABLE 9

| | | Time | | |
|---|---|---|---|---|
| | | 0 | 204 | 408 |
| 50 mM Anameg-7 | Slope | 4.73 | 60.39 | 67.68 |
| & 50 mM NGP | Intercept | 170.72 | 106.61 | 90.71 |
| 50 mM Cyglu-3 & | Slope | 14.3 | 56.8 | 66.3 |
| 50 mM NGP | Intercept | 111.0 | 112.3 | 97.8 |

TABLE 10

| | | Time | | |
|---|---|---|---|---|
| | | 0 | 204 | 408 |
| no surfactant | Slope | 4.6 | 25.1 | 37.7 |
| | Intercept | 39.3 | 92.6 | 78.6 |
| 100 mM Anameg-7 | Slope | 29.2 | 75.8 | 76.9 |
| | Intercept | 61.3 | 82.2 | 86.8 |
| 100 mM Cyglu-3 | Slope | 20.7 | 66.5 | 66.6 |
| | Intercept | 49.7 | 10.1 | 28.6 |
| 100 mM NGP | Slope | 30.2 | 81.1 | 79.7 |
| | Intercept | 43.5 | 18.2 | 41.6 |

TABLE 11

| | | Time | | |
|---|---|---|---|---|
| | | 0 | 204 | 408 |
| 50 mM octyl b-D- | Slope | 13.10 | 47.52 | 53.57 |
| galactopyranoside | Intercept | 90.69 | 73.93 | 90.91 |
| 50 mM heptyl b-D- | Slope | 28.1 | 57.0 | 40.0 |
| thioglucopyranoside | Intercept | 16.8 | 16.9 | 140.3 |
| 50 mM Anameg-7 | Slope | 21.0 | 53.6 | 59.5 |
| | Intercept | 80.7 | 117.1 | 122.0 |
| 50 mM HeptGP | Slope | 8.4 | 15.9 | 40.1 |
| | Intercept | 89.2 | 175.5 | 104.4 |
| 50 mM OGP | Slope | 27.9 | 71.5 | 65.4 |
| | Intercept | 38.8 | 4.4 | 76.2 |
| Blank (no surfactant) | Slope | 0.2 | 7.6 | 14.5 |
| | Intercept | 69.7 | 137.2 | 140.5 |
| 100 mM NOGA | Slope | 14.3 | 56.8 | 66.3 |
| | Intercept | 111.0 | 112.3 | 97.8 |
| Blank (no surfactant) | Slope | 2.9 | 7.1 | 11.3 |
| | Intercept | 39.8 | 121.8 | 140.0 |
| 50 mM OMP | Slope | 15.2 | 30.1 | 48.6 |
| | Intercept | 87.5 | 202.2 | 166.9 |
| 100 mM OMP | Slope | 43.0 | 71.5 | 79.0 |
| | Intercept | 1.9 | 90.4 | 81.0 |
| 200 mM OMP | Slope | 19.0 | 90.2 | 97.9 |
| | Intercept | 72.8 | −5.2 | −10.5 |
| 50 mM OYGP | Slope | 12.5 | 12.9 | 25.4 |
| | Intercept | 78.2 | 204.9 | 173.0 |
| 100 mM OYGP | Slope | 18.8 | 39.5 | 64.2 |
| | Intercept | 94.3 | 163.9 | 80.5 |
| 200 mM OYGP | Slope | 25.0 | 68.5 | 81.0 |
| | Intercept | 70.2 | 53.9 | 16.1 |
| 50 mM OGP | Slope | 23.4 | 35.6 | 43.7 |
| | Intercept | 31.3 | 128.9 | 150.3 |
| 100 mM OGP | Slope | 33.5 | 69.9 | 82.8 |
| | Intercept | 9.6 | 76.8 | 55.6 |
| 200 mM OGP | Slope | 29.1 | 75.1 | 90.3 |
| | Intercept | 46.5 | 34.8 | −11.0 |
| Blank (no surfactant) | Slope | 5.9 | 17.4 | 24.2 |
| | Intercept | 30.6 | 91.4 | 105.2 |

TABLE 12

| | | Time | | |
|---|---|---|---|---|
| | | 0 | 204 | 408 |
| 50 mM Anameg-5 | Slope | 12.16 | 26.97 | 36.43 |
| | Intercept | 38.77 | 66.40 | 63.17 |
| 100 mM Anameg-5 | Slope | 9.7 | 31.8 | 47.1 |
| | Intercept | 45.2 | 47.2 | 18.0 |
| 200 mM Anameg-5 | Slope | 11.9 | 28.0 | 43.7 |
| | Intercept | 51.6 | 97.9 | 68.6 |
| 50 mM Anameg-6 | Slope | 11.7 | 22.4 | 28.2 |
| | Intercept | 54.5 | 108.2 | 132.8 |
| 100 mM Anameg-6 | Slope | 15.2 | 35.3 | 44.7 |
| | Intercept | 60.2 | 156.0 | 183.0 |
| 200 mM Anameg-6 | Slope | 18.6 | 74.7 | 94.8 |
| | Intercept | 63.6 | 21.7 | −18.4 |
| 50 mM Anameg-7 | Slope | 33.0 | 56.5 | 70.9 |
| | Intercept | 32.9 | 112.0 | 94.0 |
| 100 mM Anameg-7 | Slope | 19.4 | 59.1 | 66.3 |
| | Intercept | 98.3 | 68.4 | 73.2 |
| 200 mM Anameg-7 | Slope | 20.9 | 78.0 | 92.1 |
| | Intercept | 77.2 | 48.1 | 21.9 |
| 50 mM Anameg-8 | Slope | 18.2 | 28.3 | 38.7 |
| | Intercept | 64.3 | 150.9 | 153.9 |
| 100 mM Anameg-8 | Slope | 30.2 | 76.2 | 83.0 |
| | Intercept | 56.8 | 75.2 | 68.7 |
| 200 mM Anameg-8 | Slope | 35.6 | 89.1 | 86.0 |
| | Intercept | 19.3 | −19.9 | 22.6 |
| 50 mM Anameg-9 | Slope | 25.0 | 32.5 | 40.7 |
| | Intercept | −6.5 | 150.9 | 176.9 |
| 100 mM Anameg-9 | Slope | 30.1 | 78.3 | 96.0 |
| | Intercept | 49.9 | 42.7 | −9.4 |
| 200 mM Anameg-9 | Slope | 19.4 | 59.1 | 66.3 |
| | Intercept | 98.3 | 68.4 | 73.2 |
| Blank (no added | Slope | 5.7 | 23.1 | 28.2 |
| surfactant) | Intercept | 42.8 | 77.8 | 95.6 |

TABLE 13

| | | Time | | |
|---|---|---|---|---|
| | | 0 | 204 | 408 |
| 50 mM beta-cyglu-1 | Slope | 7.6 | 21.4 | 25.6 |
| | Intercept | 51.6 | 77.4 | 89.1 |
| 100 mM beta-cyglu-1 | | | | |
| | Slope | 10.4 | 25.4 | 31.9 |
| | Intercept | 35.8 | 60.3 | 58.1 |
| 200 mM beta-cyglu-1 | | | | |
| | Slope | 7.8 | 32.9 | 44.7 |
| | Intercept | 75.2 | 66.2 | 57.9 |
| 50 mM beta-cyglu-2 | | | | |
| | Slope | 7.8 | 26.1 | 37.9 |
| | Intercept | 91.1 | 118.2 | 120.2 |
| 100 mM beta-cyglu-2 | | | | |
| | Slope | 10.1 | 35.4 | 44.5 |
| | Intercept | 89.5 | 130.8 | 156.2 |
| 200 mM beta-cyglu-2 | | | | |
| | Slope | 15.4 | 46.6 | 62.8 |
| | Intercept | 72.9 | 124.6 | 105.8 |
| 50 mM beta-cyglu-3 | | | | |
| | Slope | 16.9 | 27.3 | 40.0 |
| | Intercept | 71.7 | 209.0 | 212.4 |
| 100 mM beta-cyglu-3 | | | | |
| | Slope | 18.4 | 47.6 | 64.0 |
| | Intercept | 105.5 | 205.2 | 155.3 |
| 200 mM beta-cyglu-3 | | | | |
| | Slope | 9.7 | 47.7 | 64.2 |
| | Intercept | 107.8 | 114.6 | 64.1 |
| 50 mM alpha-cyglu-3 | | | | |
| | Slope | 16.1 | 32.1 | 42.8 |
| | Intercept | 76.1 | 174.3 | 189.4 |
| 100 mM alpha-cyglu-3 | | | | |
| | Slope | 18.5 | 55.3 | 62.5 |
| | Intercept | 89.7 | 150.2 | 156.8 |
| 200 mM alpha-cyglu-3 | | | | |
| | Slope | 21.5 | 60.9 | 77.2 |
| | Intercept | 46.6 | 57.8 | 29.2 |
| blank (no surfactant) | | | | |
| | Slope | 6.2 | 12.0 | 21.1 |
| | Intercept | 37.0 | 131.4 | 127.3 |

TABLE 14

| | | Time | | |
|---|---|---|---|---|
| | | 0 | 204 | 408 |
| TC: standard mix, | Slope | 16.27 | 66.87 | 71.66 |
| 100 mM Anameg-7 | Intercept | 85.19 | 38.55 | 27.93 |
| TC: standard mix, | Slope | −2.6 | 48.4 | 55.9 |
| 100 mM Cyglu-3 | Intercept | 118.5 | 74.9 | 85.4 |
| TC: standard mix, | Slope | 18.1 | 45.9 | 53.1 |
| 100 mM NGP | Intercept | 71.0 | 161.8 | 150.3 |
| TC: standard mix, | Slope | 0.0 | 11.5 | 19.0 |
| no added | Intercept | 82.7 | 134.6 | 139.1 |
| surfactant | | | | |
| TC: 2% BSA, 100 mM | Slope | 8.0 | 46.9 | 60.5 |
| Anameg-7 | Intercept | 133.9 | 174.4 | 156.5 |
| TC: 2% BSA, 100 mM | Slope | 21.6 | 42.8 | 51.1 |
| NGP | Intercept | 50.4 | 151.3 | 165.5 |
| TC: 2% BSA, no | Slope | 4.5 | 19.3 | 22.2 |
| added surfactant | Intercept | 66.3 | 113.5 | 141.7 |
| TC: diaphorase, | Slope | 24.8 | 57.8 | 61.3 |
| 100 mM Anameg-7 | Intercept | 70.2 | 119.9 | 119.8 |
| TC: diaphorase, | Slope | 20.8 | 56.4 | 63.5 |
| 100 mM Cyglu-3 | Intercept | 57.0 | 27.7 | 23.7 |
| TC: diaphorase, | Slope | 33.3 | 55.2 | 59.3 |
| 100 mM NGP | Intercept | 23.9 | 111.2 | 107.5 |
| TC: diaphorase, no | Slope | 4.2 | 8.7 | 10.6 |
| added surfactant | Intercept | 66.2 | 151.7 | 185.5 |
| TC: ChE, 100 mM | Slope | 23.2 | 49.5 | 58.9 |
| Anameg-7 | Intercept | 65.6 | 149.0 | 123.0 |
| TC: ChE, 100 mM | Slope | 33.5 | 59.3 | 77.3 |
| Cyglu-3 | Intercept | 60.0 | 107.6 | 52.5 |
| TC: ChE, 100 mM | Slope | 5.6 | 37.1 | 51.2 |
| NGP | Intercept | 138.0 | 192.4 | 158.3 |
| TC: ChE, no added | Slope | 5.5 | 18.5 | 19.6 |
| surfactant | Intercept | 45.4 | 61.1 | 70.9 |

TABLE 15

| | | Time | | |
|---|---|---|---|---|
| | | 0 | 204 | 408 |
| TRG: 1% | Slope | 8.05 | 43.28 | 51.89 |
| Anameg-7 | Intercept | 26.68 | 57.72 | 69.44 |
| TRG: 5% | Slope | 2.20 | 31.62 | 38.26 |
| Anameg-7 | Intercept | 20.72 | 51.15 | 63.83 |
| TRC: 1% | Slope | 11.3 | 61.1 | 65.5 |
| Cyglu-3 | Intercept | 25.2 | 33.5 | 48.3 |
| TRG: 5% | Slope | 4.2 | 52.0 | 63.3 |
| Cyglu-3 | Intercept | 28.5 | 59.9 | 51.6 |
| TRG: 1% | Slope | 3.4 | 46.1 | 61.7 |
| NGP | Intercept | 21.7 | 63.0 | 52.2 |
| TRG: 5% | Slope | 1.0 | 37.7 | 51.6 |
| NGP | Intercept | 20.8 | 62.1 | 65.9 |
| TRG: no | Slope | −0.9 | 0.8 | 2.7 |
| added | Intercept | 26.9 | 35.4 | 42.2 |
| surfactant | | | | |

The features disclosed in the above description, the claims and the drawings may be important both individually and in any combination with one another for implementing the invention in its various embodiments.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present invention in detail and by reference to specific embodiments thereof, it will be apparent that modification and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

1. A sensor for determining the amount of triglyceride and/or cholesterol in a sample, the sensor comprising:
(a) a surfactant of formula (1) 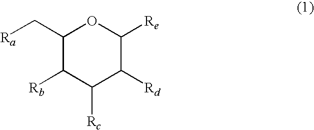
wherein
each of Rₐ, R_{b}, R_{c}, R_{d} and Rₑ is independently —OH, C₁-C₄ alkoxy or a group of formula —OCONH(CH₂)_{m′}CH₃, —OCO(CH₂)_{m′}—CH₃, O(CH₂)ₘ—CH₃,
—S(CH₂)_{m″}—CH₃, —O(CH₂)ₙ-A, —S(CH₂)ₙ-A, —OCO(CH₂)ₘ—CH₃ or
—NHCO(CH₂)ₘ—CH₃, wherein m is from 4 to 20, m′ is from 4 to 20, m″ is from 4 to 6 or from 8 to 20, n is from 0 to 10 and A is a C₃-C₈ cycloalkyl group or a phenyl group,
provided that at least one of the groups Rₐ, R_{b}, R_{c}, R_{d} and Rₑ is not —OH or C₁-C₄ alkoxy; and
(b) an enzyme reagent for measuring triglycerides and/or an enzyme reagent for measuring cholesterol.

2. The sensor according to claim 1, wherein said surfactant is of formula (I) 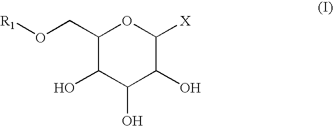
wherein:
i) R₁ is a group of formula —CONH(CH₂)_{m′}—CH₃ or —CO(CH₂)_{m′}—CH₃ wherein m′ is from 4 to 20; and X is —OH or C₁-C₄ alkoxy; or
ii) R₁ is hydrogen or C₁-C₄ alkyl; and X is a group of formula
—O(CH₂)ₘ—CH₃, —S(CH₂)_{m″}—CH₃, —O(CH₂)ₙ-A, —S(CH₂)ₙ-A,
—OCO(CH₂)ₘ—CH₃ or —NHCO(CH₂)ₘ—CH₃ wherein m is from 4 to 20, m″ is from 4 to 6 or from 8 to 20, n is from 0 to 10 and A is a C₃-C₈ cycloalkyl group or a phenyl group.

3. The sensor according to claim 2, wherein m′ is from 3 to 10.

4. The sensor according to claim 2, wherein m is from 5 to 9.

5. The sensor according to claim 2, wherein m″ is from 4 to 6.

6. The sensor according to claim 2, wherein n is from 0 to 5.

7. The sensor according to claim 2, wherein A is a C₃-C₈ cycloalkyl group.

8. The sensor according to claim 2, wherein A is cyclohexyl.

9. The sensor according to claim 2, wherein R₁ is a group of formula —CONH(CH₂)_{m′}—CH₃ or —CO(CH₂)_{m′}—CH₃ and X is —OH or C₁-C₄ alkoxy.

10. The sensor according to claim 2, wherein R₁ is hydrogen or C₁-C₄ alkyl and X is a group of formula —O(CH₂)ₘ—CH₃, —S(CH₂)_{m″}—CH₃, —OCO(CH₂)ₘ—CH₃ or —NHCO(CH₂)ₘ—CH₃.

11. The sensor according to claim 2, wherein R₁ is hydrogen or C₁-C₄ alkyl and X is a group of formula —O(CH₂)ₙ-A or —S(CH₂)ₙ-A.

12. The sensor according to claim 1, wherein the surfactant is methyl-6-O—(N-heptylcarbamoyl)-D-glucopyranoside or 3-cyclohexyl-1-propyl-D-glucoside.

13. The sensor according to claim 12, wherein the surfactant is of the formula (Ia), (Ib) or (Ic): 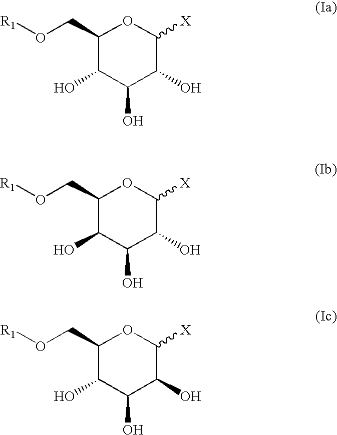
wherein R₁ and X are as defined in any one of the preceding claims.

14. The sensor according to claim 1, which sensor additionally comprises an electrochemical cell having at least two electrodes; a coenzyme, a redox agent capable of being oxidized or reduced to form a product and a reductase.

15. The sensor according to claim 14, wherein the surfactant, enzyme reagent and the coenzyme, redox agent and reductase are present as a single reagent mixture.

16. The sensor according to claim 1, wherein the enzyme reagent for measuring cholesterol comprises: (i) cholesterol esterase or a lipase; and (ii) cholesterol dehydrogenase; and/or wherein the enzyme reagent for measuring triglyceride comprises: (i) cholesterol esterase or a lipase; and (ii) glycerol dehydrogenase.

17. A method for determining the amount of cholesterol and/or triglyceride in a sample, the method comprising:
contacting the sample with a surfactant as defined in claim 1; and
determining the amount of cholesterol and/or triglyceride present.

18. The method according to claim 17, wherein the determination of the amount of cholesterol and/or triglyceride is an electrochemical determination and wherein the method optionally comprises contacting the sample with the surfactant, an enzyme reagent, a coenzyme, a redox agent and optionally a reductase, in an electrochemical cell, applying a potential across the electrochemical cell and determining the electrochemical response of the cell.

19. The method according to claim 18, wherein the electrochemical response of the cell is determined at least 1.5 minutes after contacting the sample with the surfactant.

20. The method according to claim 17, wherein the amount of surfactant which is contacted with the sample is sufficient to provide a surfactant concentration of at least 20 mM in the combination of sample and surfactant.
